(19) United States
(12) Patent Application Publication
(10) Pub. No.: US 2003/0091198 A1
(43) Pub. Date: May 15, 2003

(54) Active noise control system with a helmholtz resonator

(75) Inventor: James K. Vanderveen, Blenheim (CA)

Correspondence Address: SIEMENS CORPORATION, INTELLECTUAL PROPERTY LAW DEPARTMENT, 170 WOOD AVENUE SOUTH, ISELIN, NJ, 08830, US

(73) Assignee: Siemens VDO Automotive, Inc.

(21) Appl. No.: 10/268,130

(22) Filed: Oct. 10, 2002

Related U.S. Application Data
(60) Provisional application No. 60/336,263, filed on Nov. 15, 2001.

(51) Int. Cl. A61F 11/06; G10K 11/16; H03B 29/00

(52) U.S. Cl. 381/71.4; 381/71.5

(57) ABSTRACT

An active noise control system (26) includes an error microphone (28), a speaker (29) and an active resonator (30) which communicate with an ANC controller (32). The active resonator is a Helmholtz resonator which is coupled to an air introduction body (19). The resonance frequency of the active resonator is varied in response to the ANC controller (32) which provides logic which responds to sensor signals to eliminate low frequency noise such that the ANC system speaker (29) will effectively control and/or spectral shape the remaining noise.

The present application claims priority to U.S. Provisional Patent Application Serial No. 60/336,263, filed Nov. 15, 2001.

### BACKGROUND OF THE INVENTION

The present invention relates to a vehicle noise cancellation system, and more particularly to an active noise control system which utilizes an active resonator to minimize low frequency noise.

Manufacturers have employed active and passive methods to reduce engine noise within the passenger compartment. Such noise frequently emanates from the engine, travels through the air induction system and emanates out of an inlet of the air intake into the passenger compartment.

Active noise cancellation (ANC) systems typically utilize a speaker to create a canceling noise that attenuates engine noise. The noise created is out of phase with the engine noise and combines with this noise to result in its reduction. Generally, this noise is generated in proximity to the inlet of the air induction system. In one such system, a control unit, such as a digital signal processor, obtains data from the vehicle engine, creates a predictive model of engine noise, and thereby generates the appropriate cancellation signal based on the results of this model. This signal is then transmitted to the speaker, which transforms this signal into a canceling noise. Because the control unit may not perfectly model engine noise, an error microphone is placed in proximity to the intake of the air induction system to determine if engine noise need be further attenuated.

Small vehicles provide less packaging space for the ANC system. The ANC system on such vehicles must utilize smaller components which may result in unsatisfactory noise cancellation. Also, ANC system deficiency may be particularly pronounced in four-cylinder engines which generate low frequency noise which is difficult to cancel.

Accordingly, it is desirable to provide an effective ANC system which requires minimum packaging space for utilization in relatively small vehicles.

### SUMMARY OF THE INVENTION

The active noise control system according to the present invention is mounted adjacent an air introduction body to control engine noise which is radiated through the air introduction system. The active noise control system includes an error microphone, a speaker, and an active resonator which communicate with an ANC controller.

The active resonator is a Helmholtz resonator which is coupled to the air introduction body. The resonance frequency of the active resonator is varied in response to the ANC controller which provides logic to control the active resonator in response to sensor signals such as a cam sensor signal. The active resonator eliminates low frequency noise such that the ANC system speaker will effectively control the remaining noise.

The present invention therefore provides an effective ANC system which requires minimum packaging space for utilization in relatively small vehicles when compared to conventional passive/active noise control system for a four-cylinder application.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 is a general schematic view of an air introduction system according to the present invention; and

FIG. 2 is general graphical representation of a noise attenuation frequency band which for the present invention.

### DETAILED DESCRIPTION OF THE PREFERRED EMBODIMENT

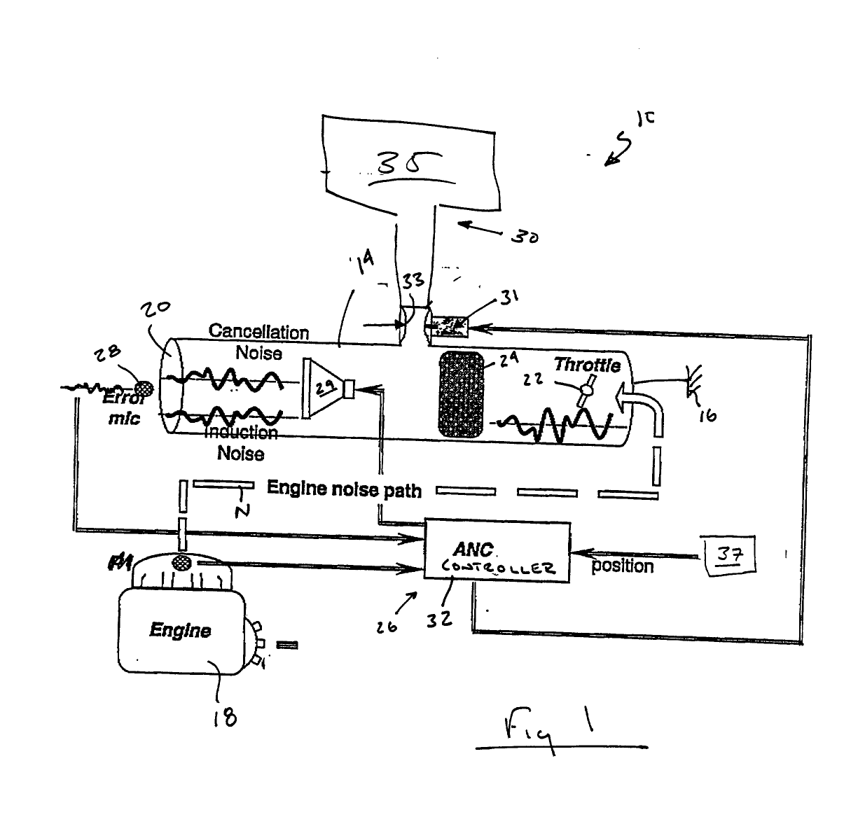

FIG. 1 illustrates a general schematic view of an air introduction system 10 for a vehicle 12. The air introduction system 10 includes an air introduction body 14 mounted adjacent a vehicle body component (illustrated schematically at 16). The air introduction body 14 intakes ambient air and communicate airflow to an engine 18. As known, noise and vibration (illustrated schematically at N) from the engine 18 emanates through the air introduction system 10 and out the inlet. The air introduction body 14 includes an inlet 20 which communicates with ambient atmosphere through a throttle 22 and an air filter 24. It should be understood that the illustrated embodiment is schematic and that other arrangements will also benefit from the present invention.

An active noise control system 26 is mounted adjacent the air introduction body 14 to control engine noise which is radiated from the air introduction system 10. The active noise control system 26 preferably includes an error microphone 28, a speaker 29 and an active resonator 30 which communicate with an ANC controller 32. The error microphone 28 is located in a reception area such that the speaker 29 of the active noise control system 26 will effectively counteract undesirable noise in response to the ANC controller 32 as generally known.

The active resonator 30 is preferably a Helmholtz resonator which is coupled to the air introduction body 14 and driven by an activator 31. A Helmholtz resonator is defined by the Helmholtz equation:

$$f = \frac{c}{2 π} \sqrt{\frac{{S}_{o} / {L}_{o}}{V}} \qquad [ 1 ]$$

where f=resonant frequency; c=sound velocity; Lₒ=length of resonating tube; Sₒ=area of resonator connecting tube; and V=volume at resonator. The active resonator 30 generally provides an air space which communicates with a Helmholtz volume 35 through an opening 33. A mass that resonates on support of a biasing force is formed by the air enclosed in the hollow space. The resonant frequency of the Helmholtz resonator depends on the area of the opening 33, on the volume of the air space, and on the length of the mass formed in the opening. When the area of the opening becomes larger, the resonance frequency is shifted toward higher frequencies. When the area of the opening is made smaller, the resonance frequency is shifted towards lower frequencies.

The resonance frequency of the active resonator 30 is varied in response to the ANC controller 32. That is, actuator 31 responds to ANC controller 32 to vary opening 33. Particularly, the ANC controller 32 provides logic which utilize a sensor 37 such as cam sensor equal to control the active resonator 30 to best match engine noise.

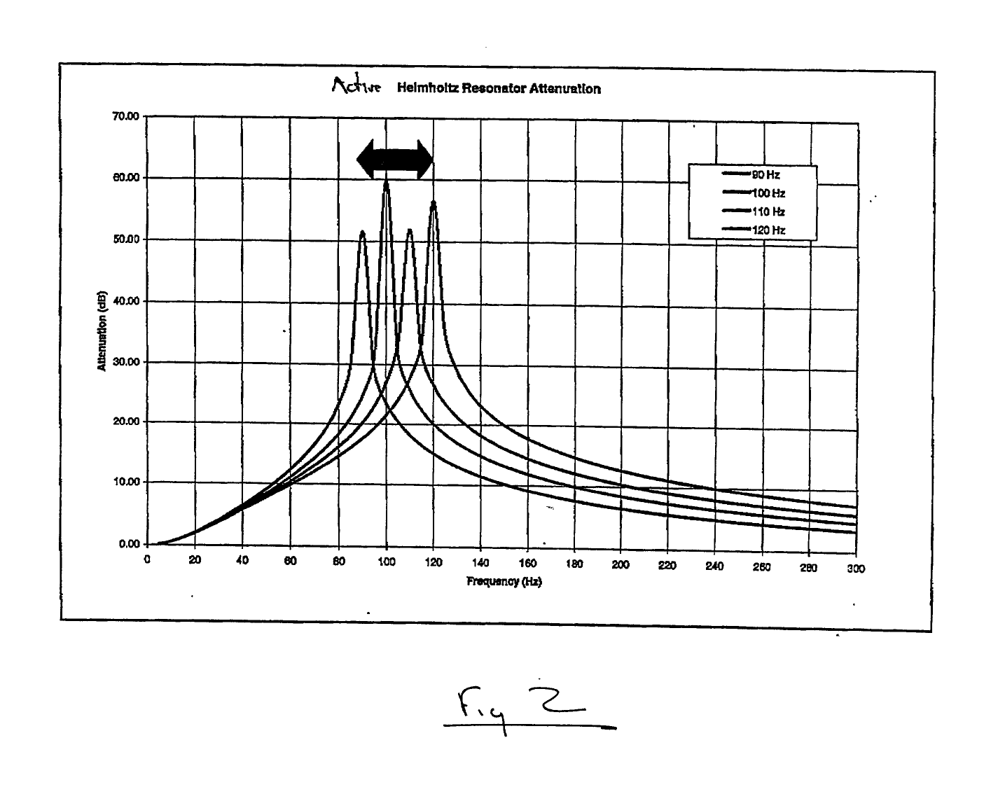

The active resonator 30 is preferably tuned to eliminate low frequency noise such that the ANC system speaker 29 will effectively control the remaining noise. For example only, an eight liter Helmholtz volume with neck length of 20mm and opening diameter range of 19-30 mm will attenuate 90 Hz-120 Hz (FIG. 2) which is an otherwise difficult frequency range for a relatively small ANC system 26 to cancel. Such a frequency range is representative of a typically four-cylinder engine, however, other ranges will also benefit from the present invention. Furthermore, the ANC system 26 and active resonator 30 will have an advantageous attenuation frequency band of 75 Hz- 150 Hz where the ANC system speaker 29 provides an attenuation capability of approximately 20 dB.

The foregoing description is exemplary rather than defined by the limitations within. Many modifications and variations of the present invention are possible in light of the above teachings. The preferred embodiments of this invention have been disclosed, however, one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

It should be understood that various sensors will benefit from the present invention.

What is claimed is:

1. An active noise control system for attenuating noise from a vehicle comprising:
an active noise cancellation controller;
an active noise cancellation speaker mounted adjacent an air introduction body, said active noise cancellation speaker in communication with said controller; and
an active resonator mounted adjacent said air introduction body, said active resonator in communication with said controller to attenuate a low frequency noise.

2. The active noise control system as recited in claim 1, further comprising an error microphone mounted adjacent said air introduction body, said error microphone in communication with said controller for a closed loop ANC system.

3. The active noise control system as recited in claim 1, wherein said active resonator has an attenuation frequency band of 90 Hz-120 Hz.

4. The active noise control system as recited in claim 1, wherein said active resonator comprises a Helmholtz resonator.

5. The active noise control system as recited in claim 1, further comprising a cam sensor in communication with said controller, said controller operable to adjust attenuation of said low frequency noise in response to said cam sensor.

6. A vehicle intake system comprising:
an air introduction body;
an active noise cancellation controller;
a cam sensor in communication with said controller;
an active noise cancellation speaker mounted adjacent said air introduction body, said active noise cancellation speaker in communication with said controller; and
a Helmholtz resonator mounted adjacent said air introduction body, said Helmholtz resonator operable to attenuate a low frequency noise in response to said controller and to said cam sensor.

7. The system as recited in claim 1, wherein said active resonator has an attenuation frequency band of 90 Hz-120 Hz.

8. A method of noise cancellation for a vehicle intake system comprising the steps of:
(1) driving an active noise cancellation speaker in response to an active noise cancellation controller which communicates with an error microphone adjacent an air introduction body;
(2) determining an engine speed; and
(3) adjusting an active resonator to attenuate a low frequency noise in response to said step (2).

9. A method as recited in claim 8, wherein said step (2) further comprising adjusting the active resonator within an attenuation frequency band of 90 Hz-120 Hz.
